(19) United States
(12) Patent Application Publication
(10) Pub. No.: US 2019/0290812 A1
(43) Pub. Date: Sep. 26, 2019

(54) FULLY DISPOSABLE SUCTION AND IRRIGATION APPARATUS FOR USE IN MEDICAL PROCEDURES

(71) Applicant: UNIVERSITY HOSPITALS CLEVELAND MEDICAL CENTER, Cleveland, OH (US)

(72) Inventors: Maroun Tanus SEMAAN, Pepper Pike, OH (US); Cliff Andrew MEGERIAN, Shaker Heights, OH (US)

(73) Assignee: University Hospitals Cleveland Medical Center, Cleveland, OH (US)

(21) Appl. No.: 15/759,676

(22) PCT Filed: Sep. 14, 2016
(86) PCT No.: PCT/US2016/051626

§ 371 Date: Mar. 13, 2018

Related U.S. Application Data
(60) Provisional application No. 62/218,034, filed on Sep. 14, 2015.

(51) Int. Cl. A61M 1/00; A61B 50/22; A61M 3/02; A61B 50/36

(52) U.S. Cl. CPC A61M 1/0064; A61B 50/22; A61M 3/0266; A61B 90/92; A61M 1/0086; A61M 3/0279; A61B 50/36

(57) ABSTRACT

A medical suction & irrigation system used for removing bodily fluid and debris from a surgical site and particularly a hand-held medical device that utilizes suction tips and suction/irrigation tips during medical procedures. The system features use of a fully disposable suction and irrigation devices. This system comprises a series of devices which includes a universal handle member, interchangeable tip members, a storage member, and a receptacle member. This system will improve the workflow of users at all skill levels while integrating seamlessly into existing common practice. It is a fully integrated and optimized system which recognizes and utilizes the interaction between two distinct user groups with the overall benefit of reducing procedural times and increasing patient safety.

### CROSS-REFERENCES TO RELATED APPLICATIONS

This application claims the benefit of U.S. provisional application Ser. No. 62/218,034 filed on Sep. 14, 2015 and incorporated herein by reference.

### BACKGROUND

During medical procedures, in particular Ear, Nose and Throat (ENT) procedures, it is common practice for a physician or other health care professional to apply suction to a surgical site with the aim of keeping the site clear of any liquids or bodily fluids that would obstruct the health care professional from viewing or effectively treating the surgical site.

As well as applying suction, it is also common practice for health care professionals to provide the surgical site with irrigation. The irrigation is needed to mix with bodily matter or debris, for example bone dust, and enable the bodily matter or debris to be suctioned away from the wound site with ease. The irrigation fluid may be water, saline, or any other fluid that will safely provide the desired functionality.

The devices used for these functions generally come in two distinct types, one of which provides a suction only function and one which provides both a suction and irrigation function from the same device. These two types of devices are called Surgical Suction Tips and Surgical Suction/Irrigator Tips respectively.

Surgical Suction Tips are generally embodied as follows: A long metal cannula is used to apply the suction to the surgical site; this long metal cannula is bent at an obtuse angle which divides the cannula into a longer section and a shorter section. The user will usually grip the shorter section. The metal cannula has a “thumb plate” attached along its short section which identifies the gripping location for the user. The “thumb plate” has a hole it its center which corresponds with a hole in the cannula. The user can control the amount of suction being utilized at the surgical site by covering or uncovering this hole in the “thumb plate”. The rear of the short section of the metal cannula features geometry which allows plastic tubing to be attached via a friction fit. This plastic tubing provides a direct fluid link to the suction system of the operating room and will suck away any fluid or debris from the surgical site and into controlled and monitored bio-material storage containers.

Surgical Suction/Irrigator Tips are generally embodied in a very similar manner; the suction cannula appears almost identical to a standard Surgical Suction Tip, however, it has a separate metal irrigation cannula that runs parallel to the suction cannula along its longer section. The suction cannula and irrigation cannula are arranged in a side-by-side relationship and they do not fluidly communicate. The rear of the irrigation cannula has geometry to allow a silicone/rubber tube to be attached via friction. The silicone/rubber tube connects to a plastic tube with a luer fitting. The plastic tube then connects to a source of irrigation, generally a standard drip bag containing saline solution.

Both Surgical Suction Tips and Surgical Suction/Irrigator Tips come in various sizes and lengths to match a variety of procedural requirements; generally, it is best for the size of the tips to be kept as small as possible as not to be a visual or physical obstruction during surgical procedures.

While applying suction, clogging of the suction cannula can often occur if a piece of debris enters the metal suction cannula and becomes lodged inside. This can happen with both Surgical Suction Tips and Surgical Suction/Irrigator Tips.

When a blockage occurs, it must be manually unblocked, meaning that the blockage needs to be cleared, using either flushing or a stylet. This blockage clearing procedure can be quite time intensive depending on whom is performing the task and their level of experience. This unblocking procedure is often carried out by a nurse who is assisting the primary physician. If the working relationship between both parties is not finely honed, then this procedure can be quite cumbersome.

The unblocking procedure usually involves a handover of the suction device from the physician to the nurse (or other assistant). The nurse must then disconnect the suction tip from the OR suction system, and the nurse must then disconnect the irrigation tip from the irrigation source. The nurse must then clear the blockage using either flushing or a stylet. Depending on how severe the blockage is, it may take a substantial amount of time, while in some cases the nurse may be unable to clear the blockage.

Once the blockage is cleared, the nurse must reconnect the suction and irrigation and then hand it back over to the physician. Meanwhile, the entire medical procedure has been held up by this event. Blockages like this can potentially occur quite often during a procedure which can add an exceptional amount of time to the overall procedure with repercussions in terms of cost and patient safety.

As well as this aspect, physicians will often require differing size tips depending on the stage of the procedure and the particular task that is being undertaken, even for a simple task like changing tip size, the same cumbersome method detailed above is required, with the same negative repercussions.

What is desired is an apparatus that simplifies these cumbersome tasks and enables nurses or other assistants of any skill level or experience level to rapidly interact with a physician and either change tip size or unblock a tip as required with minimal procedural down time. Minimizing or eliminating these hand over times will have a dramatic impact on procedure times and therefore saving money and increasing patient safety levels.

### SUMMARY

The present disclosure generally relates to medical suction & irrigation systems used for removing bodily fluid and debris from a surgical site and particularly to hand-held medical devices that utilize suction tips and suction/irrigation tips during medical procedures.

Disclosed herein is a system featuring fully disposable suction and irrigation devices. This system is currently embodied as a series of devices which includes a universal handle member, interchangeable tip members, a storage member and a receptacle member.

This system will improve the workflow of users at all skill levels while integrating seamlessly into existing common practice. It is a fully integrated and optimized system which recognizes and utilizes the interaction between two distinct user groups with the overall benefit of reducing procedural times and increasing patient safety.

Provided is a plurality of example embodiments, including, but not limited to, a system comprising: a universal handle member; a plurality of interchangeable tip members; a storage member; a receptacle member; and an unblocking member; wherein, the apparatus is configured to be fully disposable.

Also provided is a system for supporting a medical procedure, comprising: a handle member configured to attach to a source of suction at one end, and further configured with a receiving portion at another end; a plurality of tip members configured to attach to the handle member at the receiving portion, the plurality of tip members including tip members of at least two different types; a receptacle member configured to receive a used tip member in permanent and secure manner; a storage member configured for receiving and holding the plurality of tip members in a removable manner, the storage member also being configured for receiving the receptacle member.

Also provided are any of the above systems, wherein the universal handle member is configured to be connected to a source of suction.

Also provided is the above system, wherein the universal handle member is configured to be connected to a source of irrigation by having a female luer connection embodied on the universal handle member.

Also provided are any of the above systems, wherein the universal handle member is configured to be comfortably held in the hand of a physician for long periods of time, specifically during ENT procedures.

Also provided are any of the above systems, wherein the universal handle member is configured to have receiving geometry in its distal section to allow the attachment of multiple variations of interchangeable tip members.

Also provided are any of the above systems, wherein each variation has a common engagement section to allow all variations to connect to the universal handle member.

Also provided are any of the above systems, wherein the common engagement section is designed to fit securely into the universal handle member with a secure but easily releasable snap fit engagement.

Also provided are any of the above systems, wherein the universal handle member contains a check valve to only allow fluid to be released when an appropriate interchangeable tip member is connected.

Also provided are any of the above systems, wherein each variation has a unique color that matches the storage member of its origin.

Also provided are any of the above systems, wherein the storage member has a color that matches the color of the interchangeable tip members stored within it.

Also provided are any of the above systems, wherein the receptacle is configured to be connected to the storage member, but to be releasable with intention, to enable separate incineration.

Further provided are any of the above systems, wherein one or more of the components are configured to be reusable.

Also provided are any of the above systems, wherein the geometry of the handle features a bend, allowing the interchangeable tip members to be manufactured straight.

Also provided are any of the above systems, wherein the receptacle is configured to be part of the storage member, wherein they cannot be disconnected.

Further provided are any of the above systems, wherein the universal handle member is configured to have an internal continuous suction channel from the rear of the device to the very tip.

Also provided are any of the above systems, wherein the universal handle member is configured to have an internal continuous irrigation channel from the rear of the device to the very tip.

Further provided are any of the above systems, wherein the geometry that engages with the universal handle member is a soft rubber that deforms to be securely held by corresponding geometry on the universal handle member.

Also provided are any of the above systems, wherein the geometry that engages with the universal handle member is configured to have a flexible snap fit lock that is securely held by corresponding geometry on the universal handle member until a user manually releases the flexible snap fit lock.

Also provided are any of the above systems, wherein the geometry that engages with the universal handle member is configured to have solid keyed geometry which securely holds the interchangeable tip members of in the horizontal direction and can only be released when the user pulls the interchangeable tip member in either of the vertical directions.

Also provided are any of the above systems configured with a certain number of differing tip sizes and specifications to meet the requirements of particular procedures.

Still further provided are any of the above systems having any combination of the above features.

Also provided is a method of performing a medical procedure, such as by using any of the above systems, comprising the steps of:

using a suction device having a handle device and an original tip member in a medical procedure;

replacing the original tip member by performing the steps of:

removing the original tip member from the handle device,

placing the original tip member in a receptacle provided in a storage device,

selecting a replacement tip member, and

attaching the replacement tip member to the handle device; and

continuing the medical procedure using the handle device with the replacement tip member installed thereon.

Further provided is the above method wherein the replacement tip member is of a different type than the original tip member.

Also provided are any of the above methods, wherein the suction device is also configured to provide irrigation through either the original tip member or the replacement tip member or both, the irrigation for use during the medical procedure.

Also provided are any of the above methods, further comprising the step of disposing of the original tip member during or after the medical procedure.

Also provided are any of the above methods, further comprising the step of disposing of the handle device during or after the medical procedure.

Also provided are any of the above methods further comprising the step of providing the storage device with a plurality of slots each for holding a respective tip member, said storage device also being adapted for holding the receptacle that is configured for holding a plurality of used tip members in a secure manner.

Further provided are any of the above methods, wherein the step of selecting a replacement tip member includes the step of selecting the replacement tip member from a slot in the storage device.

Also provided are additional example embodiments, some, but not all of which, are described hereinbelow in more detail.

### BRIEF DESCRIPTION OF THE DRAWINGS

The features and advantages of the example embodiments described herein will become apparent to those skilled in the art to which this disclosure relates upon reading the following description, with reference to the accompanying drawings, in which:

FIG. 1 provides a schematic drawing of an example universal handle member;

FIG. 2 provides a schematic drawing of another view of the example universal handle member of FIG. 1;

FIG. 3 provides a schematic drawing of still another view of the example universal handle member of FIG. 1 showing interior structure;

FIG. 4 provides a schematic drawing of an example replaceable tip member having only a suction cannula;

FIG. 5 provides a schematic drawing of an example replaceable tip member having both a suction cannula and an irrigation cannula;

FIG. 6 provides a schematic drawing of the example universal handle member of FIG. 1 mating with the example replaceable tip member of FIG. 5;

FIG. 7 provides a schematic drawing of an example unblocking member;

FIG. 8 provides a schematic drawing of an example storage member;

FIG. 9 provides a schematic drawing of an example disposable receptacle member;

FIG. 10 provides a schematic drawing of the example storage member of FIG. 8 having received the example receptacle member of FIG. 9;

FIG. 11 provides a schematic drawing of the example storage member of FIG. 8 having received the example receptacle member of FIG. 9 with the storage member having a plurality of replaceable tip members of FIGS. 4 and 5 stored therein;

FIG. 12 provides a schematic drawing of another example handle member with replaceable tip member;

FIG. 13 provides a schematic drawing of yet another example handle member with replaceable tip member;

FIG. 14 provides a schematic drawing of still another example handle member with replaceable tip member;

FIG. 15 provides a schematic drawing of another example storage member with integral receptacle member and replaceable tip members stored therein; and

FIG. 16 provides a schematic drawing of still another example storage member with integral receptacle member and replaceable tip members stored therein.

### DETAILED DESCRIPTION OF THE EXAMPLE EMBODIMENTS

Disclosed, for example, is a fully disposable suction and irrigation apparatus for use in medical procedures. This apparatus is provided in an example embodiment as a series of devices to be used as part of a system which includes a universal handle member, interchangeable tip members of different types, a storage member, a receptacle member, and an unblocking member.

The apparatus can be used to reduce time delays during medical procedures. The ethos of this time saving apparatus is to empower all users with a structured system to improve workflow and reduce downtime.

The system is based on two distinct user groups engaging with the system simultaneously. The two distinct groups will herein be described as primary users and secondary users. The primary user will, more often than not, be a physician, such as a surgeon, for example. The secondary user will, more often than not, be an assisting nurse or other assistant who is assisting the primary user during a medical procedure being performed on a patient.

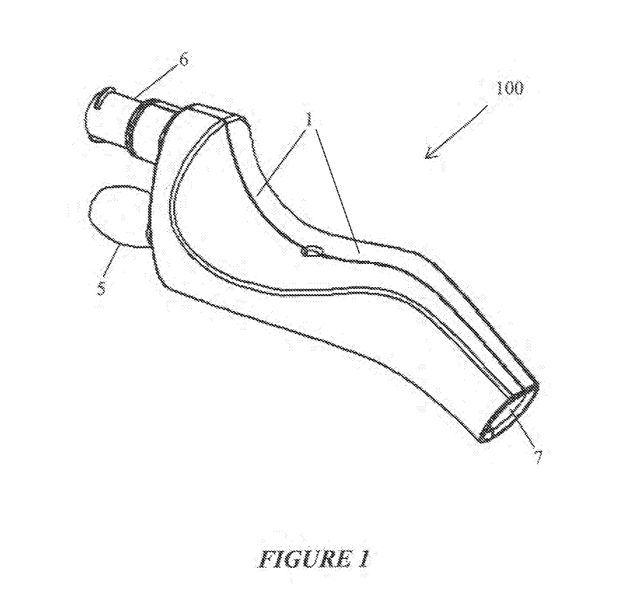

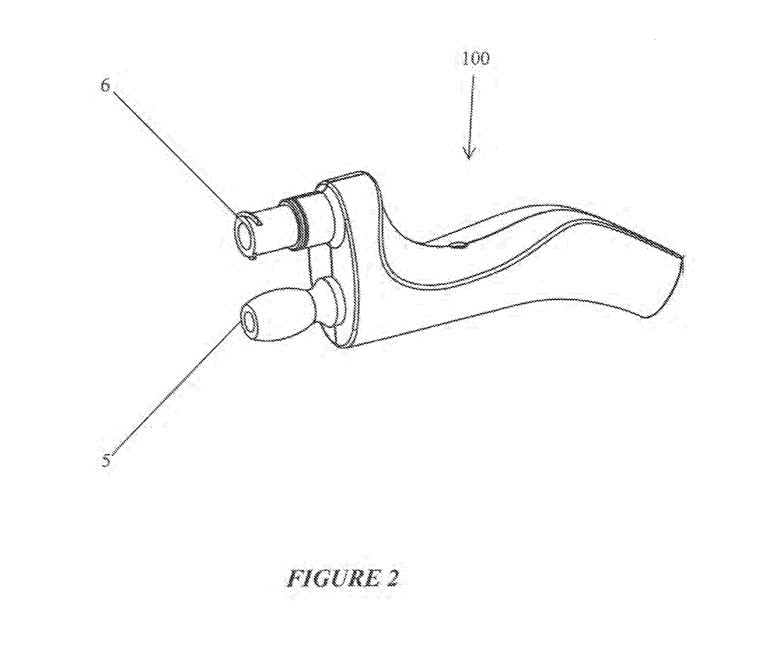

The primary user is in control and possession of a universal handle member 100 while performing the procedure. An example universal handle member is shown in an example embodiment in FIGS. 1 and 2. The universal handle member 100 is specifically designed to be comfortably used by the primary user for long periods of time without leaving his/her control. The universal handle member has a handle portion 1, and features connecting geometry 5 at the rear to allow suction tubing to be connected via friction (see the alternative view in FIG. 2). The universal handle member 100 also features a female luer connection 6 at the rear to allow an irrigation source to be connected. The universal handle member 100 also features receiving geometry 7 at the front of the handle. This receiving geometry 7 allows any type of interchangeable tip member (described below) to be connected to the handle member 100.

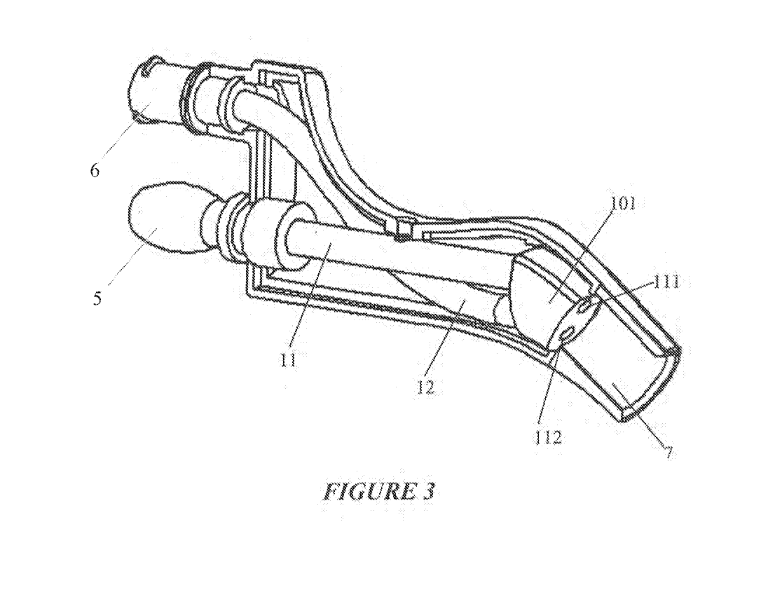

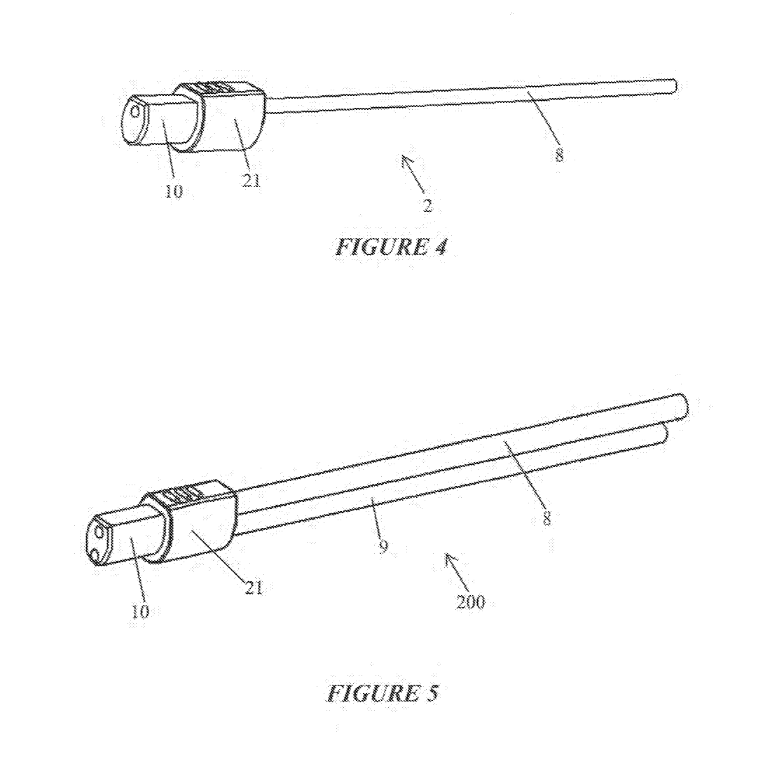

The universal handle member 100 is designed to provide separate and unbroken suction and irrigation channels, which do not fluidly communicate with each other at any stage, within the universal handle member 100 (See FIG. 3 showing an interior of the handle member). The first suction channel runs in a continuous pathway from the connecting geometry 5 at the rear of the handle, through the first internal suction channel 11, right to the header 101 having channel port 111 exiting therefrom, connecting to the tip of the suction cannula 8, when an appropriate tip member 2, 200 is attached to the universal handle member 100 (see FIGS. 4, 5). Correspondingly, the irrigation channel runs in a continuous pathway from the luer geometry 6 and the rear of the handle, through the second internal irrigation channel 12 and right to the tip of the irrigation cannula 9 via port 112, when an appropriate tip member 200 is attached to the universal handle member 100 (see FIG. 5).

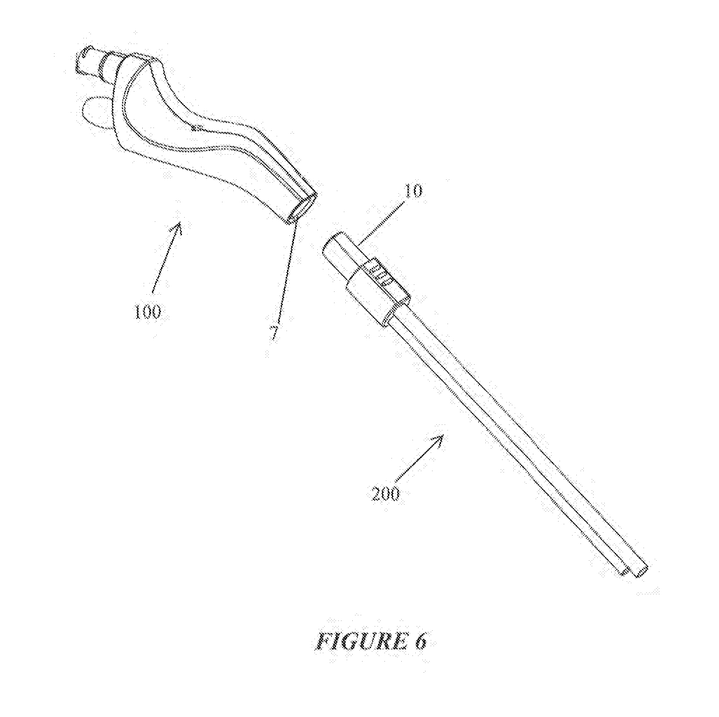

The secondary user is typically in control and possession of the storage member 3 with installed receptacle member 4 and tip members 2, 200 (see FIG. 11). The storage member is color coded, with the color matching the variation of the interchangeable tip member 2, 200 (see FIGS. 4, 5) which is held in the storage member 3. This allows for rapid recognition of tip specification by the secondary user. The storage member 3 is designed to have all tip members presented in a fashion to be easily gripped and removed from the storage member 3 for installation on the universal handle member 100 (See FIGS. 6, 11). The secondary user can also easily count how many interchangeable tip members have been used, or are in use, at any one time. Rapid recognition and presentation of the interchangeable tip members to the secondary user will increase ease of use, reduce delays, identify missing parts, and level the playing field between highly experienced secondary users and relatively novice secondary users.

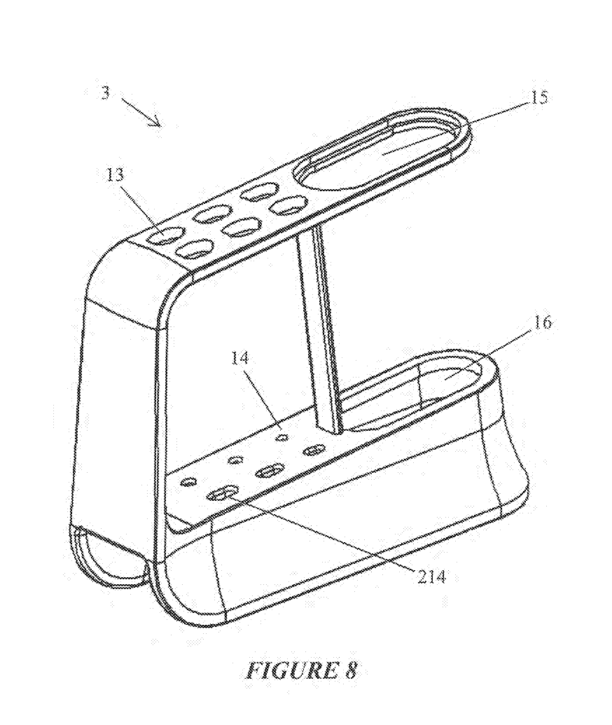

The storage member 3 typically remains under the supervision of the secondary user at all times. There could also be multiple storage members in use at any one time during a procedure (so as to hold a larger variety of tip member types and sizes, for example). The storage member 3 securely holds a multiple of interchangeable tip members 2 or 200 (See FIG. 11), or other types, at the ready. As shown in FIG. 8, the tip members 2, 200 can be held securely via specifically designed receiving geometry 13 at the top of the storage member 3, and specifically designed geometry 14, 214 at the bottom of the storage member. FIG. 11 shows the tip members 2, 200 held securely in place in a storage member 3.

The ultimate time saving benefit is achieved when a change of a tip member is required during the medical procedure. The tip member change may be because a suction cannula 8 (see FIGS. 4, 5) has become blocked, or it may be because a change of cannula or tip member size is required for procedural reasons.

In common standard practice, when a change of tip member is needed, the primary user must hand over the entire system to the secondary user, who undertakes the cumbersome process previously described. In contrast, with the system described herein, the primary user need not hand over anything; the primary user simply presents the universal handle 100, with the interchangeable tip member 2, 200 attached, to the secondary user. The secondary user then simply removes the interchangeable tip member 2, 200 from the universal handle member 1, and replaces it with the desired interchangeable tip member 2, 200 (or other). The system does not require any disconnection or reconnection of suction or irrigation tubes. The secondary user then disposes of the used tip member by placing it in the receptacle member 4 found installed in the storage member 3, without the struggle of trying to unblock the metal suction cannula 8. This greatly improves on the entire tip replacement process.

The interchangeable tip members 2, 200 (or other types and sizes) are part of the overall system. Each specification of interchangeable tip member 2, 200 (or others) can be provided as suction only, as in tip member 2 having a suction cannula 8 (see FIG. 4) that can be provided in various sizes, and/or suction/irrigation tip members 200 having both a suction cannula 8 and an irrigation cannula 9 (see FIG. 5) also provided in various sizes. The tip members 2, 200 have a common engagement section 10 to engage with handle 100 at the proximal end at the receiving geometry 7 that allows all variations of interchangeable tip members 2, 200 (among others) to engage easily with the universal handle member 100 as shown in FIG. 6 with engagement section 10 of the tip member 200 mating with receiving geometry 7 of the universal handle member 100. Tip member 2 would be similarly attached. As well as this, each different interchangeable tip member 2, 200 is color coded to match the storage member 3 it originated from, allowing for rapid recognition and identification in the overall system (see FIG. 11).

The common engagement section 10 of each tip member 2, 200 is designed to fit securely into the universal handle member 100 via engagement section 10 and receiving geometry 7 with a secure but easily releasable snap fit engagement, such as by using tabs (ribs) and indentations, for example. The secure engagement between the interchangeable tip members 2, 200 and the universal handle member 100 can be confirmed to the user with an audible click to indicate proper engagement, for example.

In an exemplary embodiment of this apparatus, the connection of a suction/irrigation interchangeable tip member 200 will result in the engagement with an internal check valve provided within header 101, contained within the universal handle member 100. The check valve in the header 101 is opened when a suction/irrigation member 200 is connected to the universal handle member 100, otherwise it remains closed. When a suction only tip member 2 is connected, this valve remains closed.

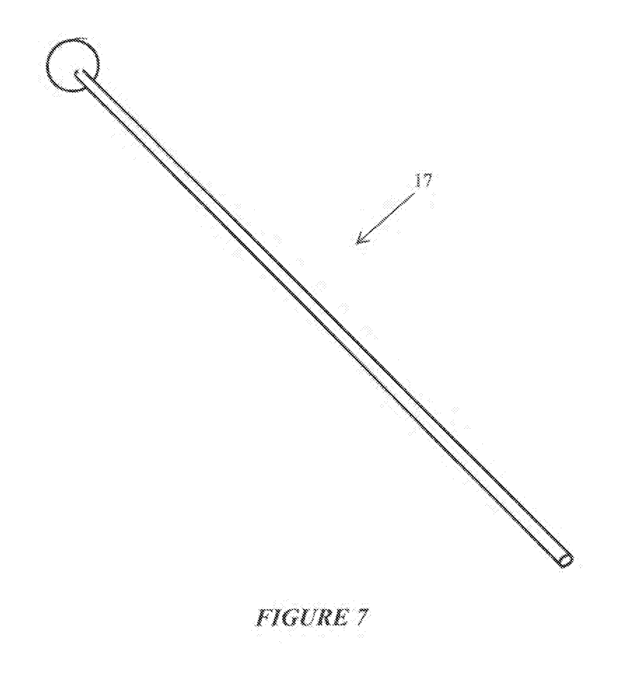

An unblocking member 17 can also be provided as part of the apparatus described herein, as shown in FIG. 7. The unblocking member 17 can take the form of a standard stylet which will come in varying working lengths and working diameters depending on the designated procedural purpose of the kit is it provided width. However, the design of the system reduced the need for such an unblocking member, as replacement of the blocked tip member is the typical response to a blockage during the medical procedure.

To aid in unblocking the cannulas, each cannula opening will be color coded, so the openings facing the secondary user in the interchangeable tip members will show a color coding, to allow secondary users to rapidly identify which opening links to each cannula.

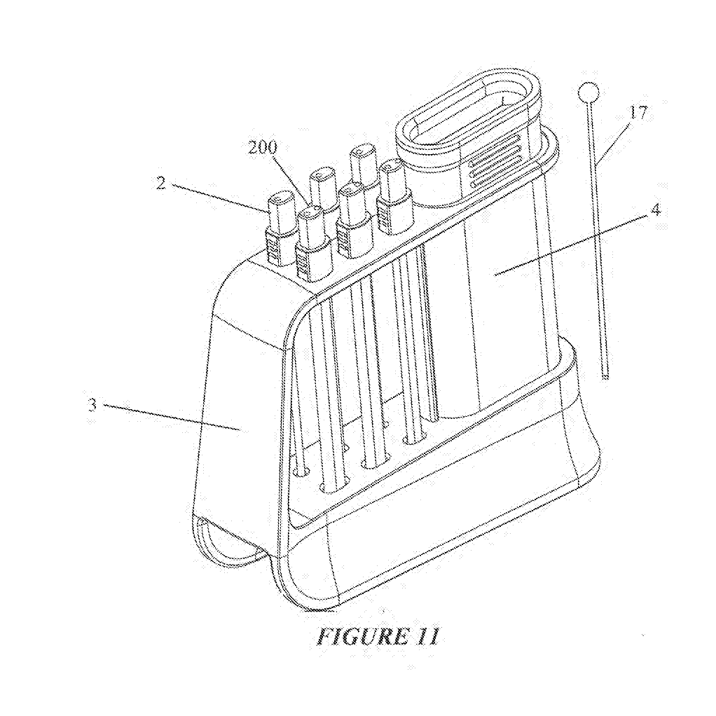

The storage member 3 also securely holds the receptacle member 4 in a similar manner to how the interchangeable tip members 2 are held and secured, as seen in FIG. 10. There is specifically designed holding geometry 15 at the top of the storage member 3, and specifically designed holding geometry 16 at the bottom of the storage member 3 (see FIG. 8).

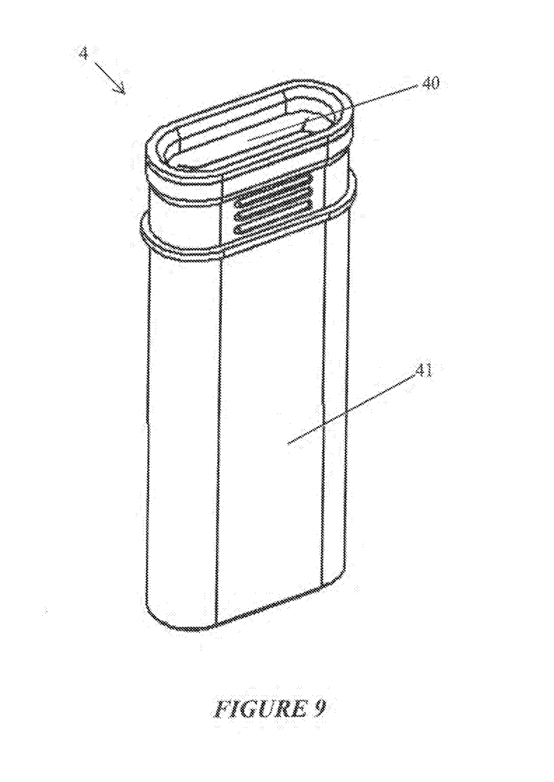

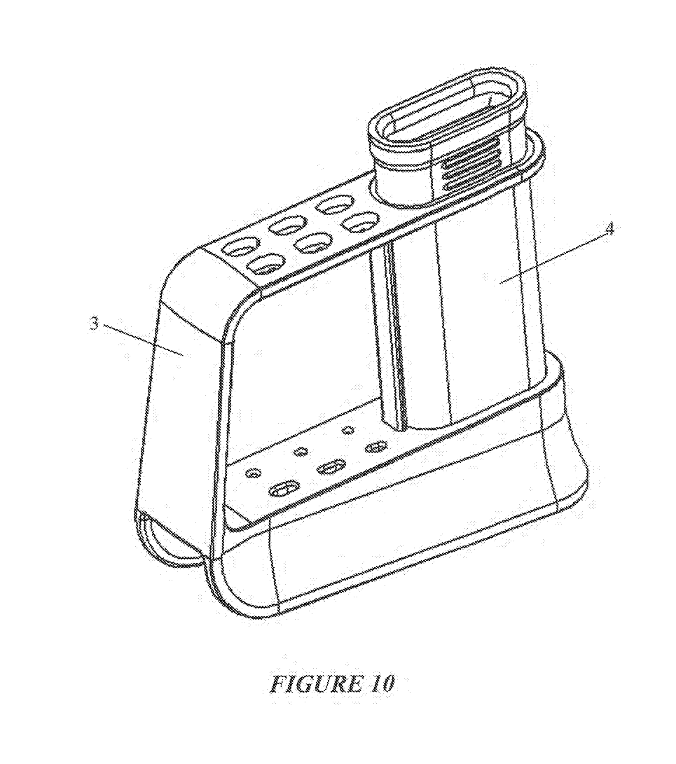

The receptacle member 4 as shown in the example of FIG. 9 is designed to receive any used interchangeable tip members 2, 200 (or others) in a chamber formed within the main body 41. The receptacle member 4 has a transparent main body 41 to allow users to track and count how many tip members are in the receptacle. The top of the receptacle member 4 features a silicone seal 40. The silicone seal 40 allows the disposable tip members to be placed into the receptacle, but will not allow the tip members to come back out again. The receptacle member 4 can easily be detached and removed from the storage member 3 for separate incineration, if desired, for sanitary reasons.

FIG. 11 shows the entire system arranged in a manner for use, with storage member 3, the receptacle member 4, and various tip members 2, 200 being held therein, along with the unblocking member 17 in proximity.

In another exemplary embodiment of the storage member (not shown), the storage member is pre-loaded with a particular selection of varying interchangeable tip members to be used in a certain procedure, for example a middle ear or temporal bone case. In these cases a selection of three 3fr, one 5fr and one 7fr would be an excellent selection. Two unblocking members would be provided with this particular arrangement, of two differing specifications, one to match the 3fr tip and one to match the 5fr and 7fr tip respectively.

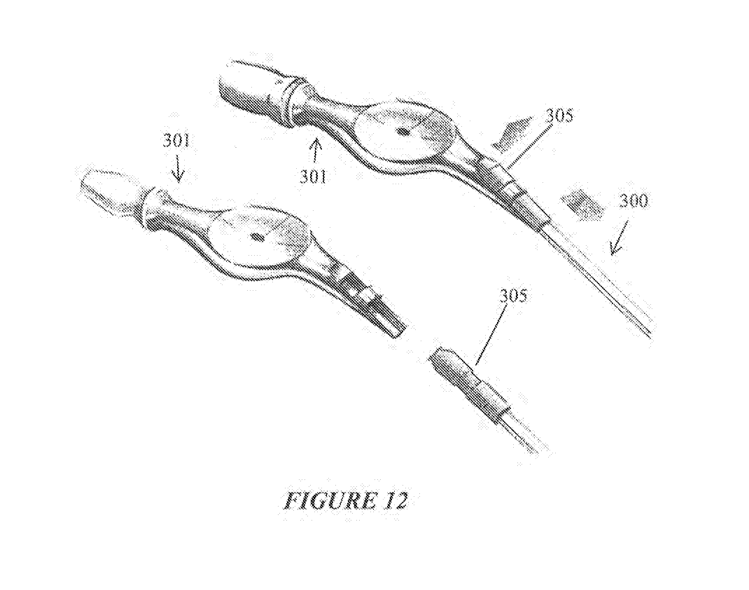

Another example embodiment for attaching interchangeable tip members 300 to a universal handle member 301 is shown in FIG. 12. In this embodiment, the connecting geometry is formed from a soft rubber that will deform in shape to allow secure fixing to the universal handle member 301. To disconnect, a user is required to squeeze the rubber section 305 at the tip while pulling the interchangeable tip members 300 in order to release it from the universal handle member 301.

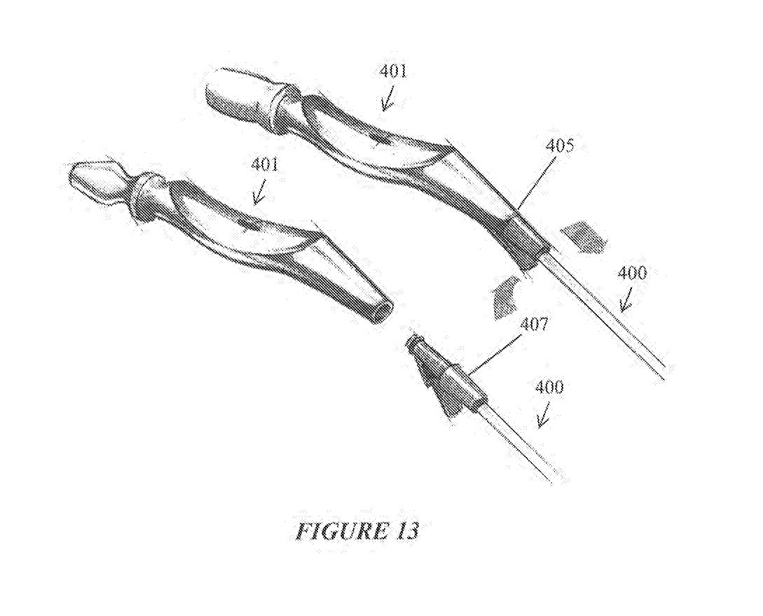

Another example embodiment for attaching interchangeable tip members 400 to the universal handle member 401 is shown in FIG. 13. In this embodiment, the connecting geometry features a flexible snap fit lock 405 designed to securely hold the interchangeable tip members 400 to the universal handle member 401. The user is required to squeeze the protruding arm 407 on the interchangeable tip member 400 to release the snap fit lock while retracting the interchangeable tip member 400.

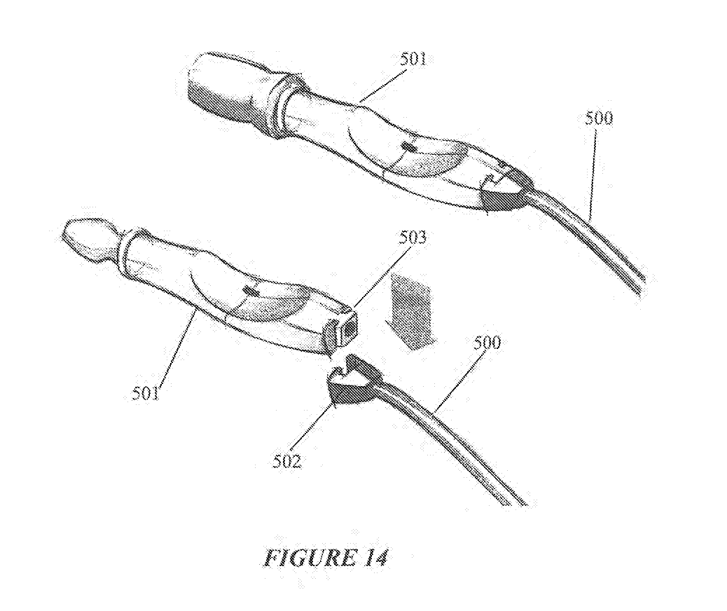

Still another example embodiment for attaching the interchangeable tip members 500 to the universal handle member 501 is shown in FIG. 14. In this embodiment the connecting geometry is designed with specific keyed geometry 502, 503 to securely hold the interchangeable tip member 500 to the universal handle member 501. The keyed geometry is only releasable when the interchangeable tip member 500 is strongly pulled in either of the vertical directions.

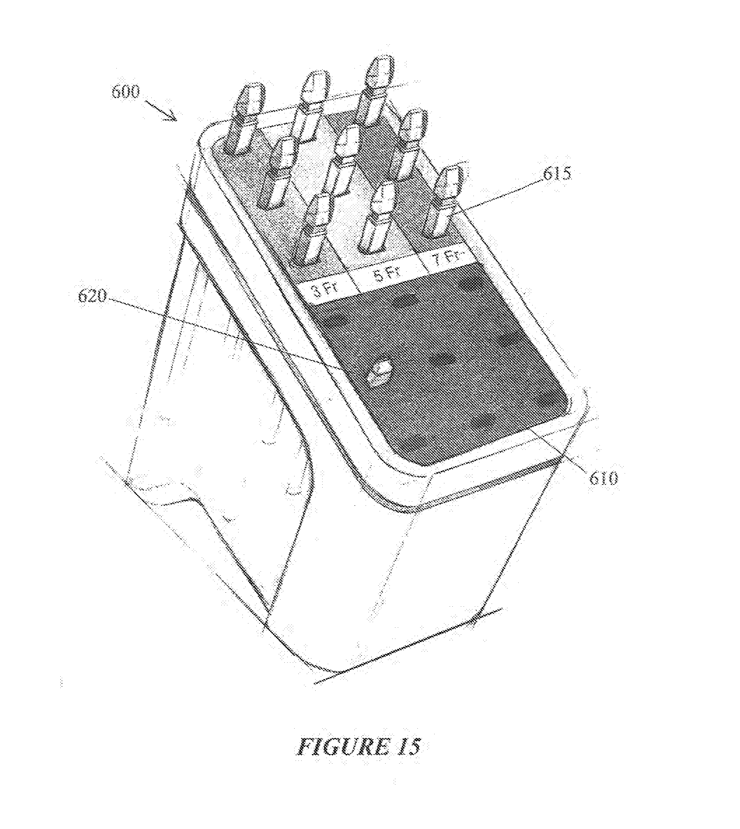

Another example embodiment of a storage member 600 is shown in FIG. 15. In this embodiment, the storage member 600 has a receptacle member 610 that are combined into a single unit, which does not disassemble. Tip members 615 are taken from their initial position, and as they are used they are moved into a specific holding position 620 that indicates that tip has been used. When a procedure is complete, the combined unit is disposed of in its entirety.

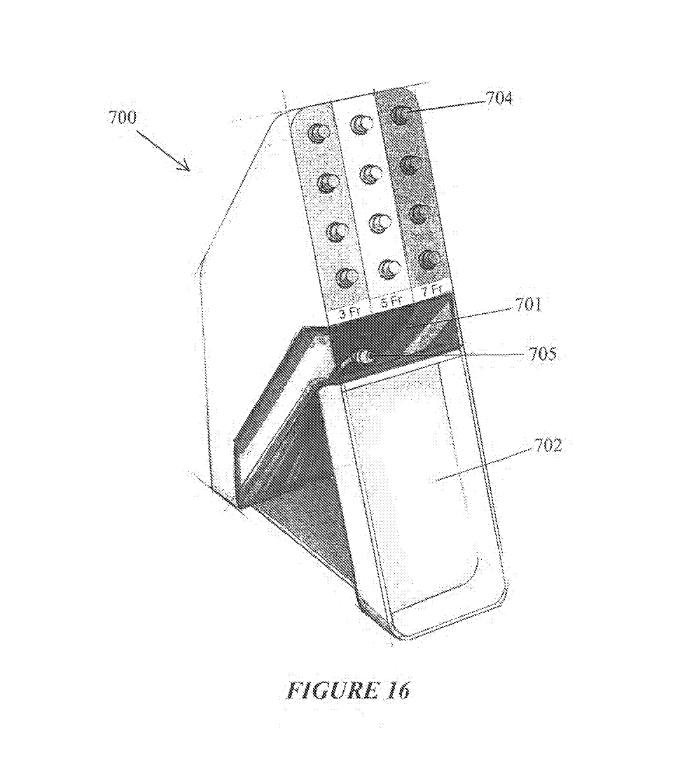

Still another example embodiment of a storage member 700 is shown in FIG. 16. In this embodiment the storage member 700 and the receptacle member 701 are combined into a single unit which does not disassemble and holds tip members 704. In this embodiment, the used interchangeable tip members 705 are placed into the transparent receptacle 701 at the base of the unit. The entire unit features a lid 702 which when opened can be utilized as a stabilizing member that can allow the quite narrow unit to sit on the sterile field and be taped into position, if needed.

The various components can be constructed of appropriate medical grade materials, such as plastics, or metals, for example. Where strength and sterilizability is most desirable, such as in the tip member (particularly the cannula portion), stainless steel might be used, or some other metal, whereas the engagement section may be plastic. The handle member, in contrast, might be made of a metal or plastic. The storage and receptacle members might be made of a plastic that can be sterilized, for example, and rubber can be used for seals or other portions, such as connectors. In particular where disposability is desired, plastic parts, where practical, can save costs.

Table 1, provided below, shows some example materials for the various components of the system of a preferred embodiment.

TABLE 1

| Numerical | | |
|---|---|---|
| Reference | Part Description | Material |
| #1 | Handle Portion | Plastic - Polypropylene (PP) |
| #3 | Storage Member | Plastic - Polypropylene (PP) |
| #4 | Receptacle Member | Plastic - Polystyrene (PS) |
| #5 | Connecting geometry | Plastic - Polypropylene (PP) |
| #6 | Female luer connection | Plastic - Clear Polyvinylchloride |
| | | (PVC) |
| #8 | Suction Cannula | Metal - Stainless Steel |
| #9 | Irrigation Cannula | Metal - Stainless Steel |
| #11 | First Internal Suction | Metal - Stainless Steel |
| | Channel | |
| #12 | Second Internal Suction | Plastic - Polyvinylchloride (PVC) |
| | Channel | |
| #17 | Unblocking Member | Metal - Stainless Steel |
| #41 | Transparent Body | Plastic - Clear Polystyrene (PS) |
| #101 | Header | Plastic - TPE |

The combination of the elements described herein, creates an apparatus and system of use that should improve the workflow of users at all skill levels while integrating seamlessly into existing common practice. It is a fully integrated and optimized system which recognizes and utilizes the interaction between two distinct user groups with the overall benefit of reducing procedural times and increasing patient safety.

Many other example embodiments can be provided through various combinations of the above described features. Although the embodiments described hereinabove use specific examples and alternatives, it will be understood by those skilled in the art that various additional alternatives may be used and equivalents may be substituted for elements and/or steps described herein, without necessarily deviating from the intended scope of the application. Modifications may be necessary to adapt the embodiments to a particular situation or to particular needs without departing from the intended scope of the application. It is intended that the application not be limited to the particular example implementations and example embodiments described herein, but that the claims be given their broadest reasonable interpretation to cover all novel and non-obvious embodiments, literal or equivalent, disclosed or not, covered thereby.

What is claimed is:

1. A system for supporting a medical procedure, comprising:
a handle member configured to attach to a source of suction at one end, and further configured with a receiving portion at another end;
a plurality of tip members configured to attach to the handle member at the receiving portion, the plurality of tip members including tip members of at least two different types;
a receptacle member configured to receive a used tip member in permanent and secure manner;
a storage member configured for receiving and holding the plurality of tip members in a removable manner, said storage member also being configured for receiving the receptacle member.

2. The system of claim 1, wherein the universal handle member includes a female luer connection at the one end that is configured to be connected to a source of irrigation.

3. The system of claim 2, wherein at least one of said plurality of tip members has both a channel for transmitting suction and also a channel for transmitting irrigation.

4. The system of claim 2, wherein the universal handle member includes a check valve to only allow fluid from the source of irrigation to be transmitted through the handle member when an appropriate interchangeable tip member is connected.

5. The system of claim 2, wherein the universal handle member includes an internal continuous irrigation channel connecting the source of irrigation attached to the luer connection to a tip member installed at the receiving portion.

6. The system of claim 5, wherein the universal handle member also includes an internal continuous suction channel connecting the source of suction attached to the one end to a tip member installed at the receiving portion.

7. The system of claim 1, wherein the universal handle member is configured of a size to be comfortably held in the hand of a physician for long periods of time.

8. The system of claim 1, wherein the receiving portion of the universal handle member is configured to allow the attachment of multiple variations of interchangeable tip members.

9. The system of claim 8, wherein each variation of the tip members has a common engagement section to allow all variations to connect to the universal handle member at the receiving portion.

10. The system of claim 9, wherein the common engagement section is designed to mate with the receiving portion in a secure but easily releasable snap fit engagement.

11. The system of claim 1, wherein the universal handle member includes a check valve to only allow fluid to be released when an appropriate interchangeable tip member is connected.

12. The system of claim 1, wherein the plurality of tip members have indication that identifies the storage member of origin.

13. The system of claim 12, wherein the storage member and the plurality of tip members are provided with a common matching color for providing said identification.

14. The system of claim 1, wherein the receptacle is provided with transparent walls so that tip members received therein may be seen from outside of the receptacle.

15. The system of claim 1, wherein the geometry of the handle member includes a bend allowing the interchangeable tip members to be manufactured straight.

16. The system of claim 1, wherein the receptacle is configured to be removable from said storage member.

17. The system of claim 1, wherein the universal handle member includes an internal continuous suction channel connecting the source of suction attached to the one end to a tip member installed at the receiving portion.

18. A method of performing a medical procedure, comprising the steps of:
using a suction device having a handle device and an original tip member in a medical procedure;
replacing the original tip member by performing the steps of:
removing the original tip member from the handle device,
placing the original tip member in a receptacle provided in a storage device,
selecting a replacement tip member, and
attaching the replacement tip member to the handle device; and
continuing the medical procedure using the handle device with the replacement tip member installed thereon.

19. The method of claim 18, wherein the replacement tip member is of a different type than the original tip member.

20. The method of claim 18, wherein the suction device is also configured to provide irrigation through either the original tip member or the replacement tip member or both, said irrigation for use during said medical procedure.

21. The method of claim 18, further comprising the step of disposing of the original tip member during or after said medical procedure.

22. The method of claim 18, further comprising the step of disposing of the handle device during or after said medical procedure.

23. The method of claim 18, further comprising the step of providing the storage device with a plurality of slots each for holding a respective tip member, said storage device also being adapted for holding the receptacle that is configured for holding a plurality of used tip members in a secure manner.

24. The method of claim 23, wherein said step of selecting a replacement tip member includes the step of selecting said replacement tip member from a slot in the storage device.
